(19) United States
(12) Patent Application Publication
(10) Pub. No.: US 2008/0003610 A1
(43) Pub. Date: Jan. 3, 2008

(54) METHOD FOR IDENTIFYING GERMS

(76) Inventors: Michael FRANK, Dusseldorf (DE); Helmut HERRMANN, Hanau (DE); Friedhelm SIEPMANN, Essen (DE); Stefan STUMPE, Dusseldorf (DE); Andreas THUNCHEN, Wuppertal (DE)

Correspondence Address: PAUL & PAUL, 2000 MARKET STREET, PHILADELPHIA, PA, 19103-3229, US

(21) Appl. No.: 11/835,713

(22) Filed: Aug. 8, 2007

Related U.S. Application Data
(63) Continuation of application No. PCT/EP2006/000473, filed on Jan. 20, 2006.

(30) Foreign Application Priority Data
Feb. 10, 2005 (DE) 10 2005 006 237.7

(51) Int. Cl. C12Q 1/68; G01N 33/554; C12Q 1/04

(52) U.S. Cl. 435/6; 435/34; 435/7.32

(57) ABSTRACT

The invention relates to a method for quantitatively and/or qualitatively identifying germs in a sample, which method comprises a step (a) of preparing the sample by labeling at least some of the germs present in the sample by means of at least one fluorescent marker and a step (b) involving a quantitative and/or qualitative detection and/or evaluation by recording and/or measuring fluorescence emission, wherein, in step (b), the sample containing the fluorescently labeled germs, prepared in step (a), is subjected to an excitation radiation of a defined wavelength or a defined wavelength range for a defined period of time, and the time course of the fluorescence emission radiation generated due to said excitation is recorded, so that a discrimination first, between the measured signals caused by the fluorescently labeled germs, second, possible interfering signals is made possible, thereby identifying the fluorescently labeled germs in the sample in a quantitative and/or qualitative manner. The invention also relates to a corresponding device for carrying out such a method.

### CROSS-REFERENCE TO RELATED APPLICATIONS.

This application is a continuation under 35 U.S.C. §365 U.S.C. §120 of International Application No. PCT/EP2006/000473, filed Jan. 20, 2006. This application also claims priority under 35 U.S.C. §119 of German Application No. DE 10 2005 006 237.7, filed Feb. 10, 2005.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC.

Not Applicable

### BACKGROUND OF THE INVENTION

(1) Field of the Invention

The present invention relates to a method for quantitatively and/or qualitatively identifying germs and to the application of such a method, in particular within the framework of production control and/or quality control. The present invention furthermore relates to a device for quantitatively and/or qualitatively identifying germs, in particular for carrying out the aforementioned method, and also to the use of said device, in particular for the preferably automated production control and/or quality control.

Germs—meaning, according to the invention, bacteria, yeasts and fungi—in products of any kind may, on the one hand, result in spoilage, directly affecting quality, mode of action and performance. On the other hand, germ loads which are too high and pathogenic germs, respectively, may cause infections and/or diseases. Immediate germ load detection is therefore essential, for example before a final product reaches the market.

Microbiological safety must be guaranteed for a multiplicity of substances, raw materials and products from the different areas of industry, trade, household, health, gastronomy etc. It should be noted here that the type and number of different germs such as, for example, bacteria and fungi, must be controlled within tight limits.

Control of the hygienic state of consumer goods such as food products, cosmetics, adhesives, detergents and cleaners, but also, for example, of cooling lubricants which are used in particular in the field of industry, is governed by legislation. Depending on the type of goods, different thresholds of microbiological load are defined. The same applies to surfaces in hospitals (e.g., in operating theatres), but increasingly also to air-conditioning systems, heat exchangers and the like.

There are a large variety of detection methods whose complexity, apparatus required and analysis time required usually depend on the germ contents to be detected, in particular specification of the maximum number of germs, and the matrix in which said germs can occur. In the present application, bacteria (Gram-positive and Gram-negative), yeasts and fungi in particular are referred to as germs.

For a long time, quantitative microbiological analytical techniques have been used for quality assurance. These methods are based on propagating individual germs which occur in the material to be investigated in such a way that they become visible to the naked eye as smears or colonies. Routine culturing methods used for this propagate said germs either on solid nutrient media or in liquid nutrient solutions or media. Conventional culturing methods for detecting bacteria and fungi may take up to several days, depending on the method and type of germs.

Customarily, the “most classical” method for detecting germs is the “plate culture method.” Here, the sample to be analyzed is applied to a Petri dish coated with nutrient medium (e.g., agar agar) and cultured under defined conditions for a particular time. In the case of a germ-loaded sample, colonies start to grow on the nutrient medium within one or more days, which can be detected with the naked eye and counted. This is a relatively simple but slow way of successfully detecting germs, provided that the germs to be detected are given a suitable nutrient medium and that the environmental conditions—influenced, for example, by oxygen content, temperature, light, etc.—promote growth. In this way it is possible to detect germs up to a limit of one colony-forming unit (cfu) per milliliter (ml). The relatively uncomplicated manageability of the plate culture method which, in addition, does not require any explicit microbiological specialist knowledge renders this method still a method of choice for a number of applications, even now.

(2) Description of Related Art, Including Information Disclosed Under 37 C.F.R. §§1.97 and 1.98.

According to the prior art, the culturing methods generally involve inoculating nutrient media (typically culture dishes containing nutrient media based on agar agar) with the sample and culturing them at usually elevated temperatures adapted to the particular germs for up to one week (e.g., in an incubator). A person skilled in the art is then able to derive the type and extent of the microbial load of the sample from growth and shape of the resulting cultures.

A decisive disadvantage of this technology is the fact that only an undefined fraction of the germs present in the sample can be cultured and the information is available only after one week.

In order to solve the above-described problems, a number of methods have already been developed in the past in order to accelerate microbial detection or to increase sensitivity. Said methods include microscopic methods in which germs are selectively or unselectively stained and detected accordingly, or methods based on immunoassays and direct molecular-biological methods which amplify and then gel-electrophoretically detect the idioplasm of the germs.

For some time it has been attempted to reduce detection time from a few days to a few hours or even less by new “rapid detection methods.” “Rapid detection methods” are already employed partially (impedance, bioluminescence, etc.), but there is a demand for more direct and more rapid methods. This is because the previously established “rapid detection methods” are based on a time-dependent enrichment of biological material, and thus in analysis still require from 24 to 48 hours.

Analytical methods using more complicated equipment are based, for example, on measuring conductivity in a germ-containing solution. This involves monitoring the change in conductance caused by germ growth and the metabolism-generated components of the solution. However, this impedance method of determining the alternating current resistance has the disadvantage that significant changes in conductance can be determined only with germ numbers of at least 10³ to 10⁴ germs per ml. Although it is possible also to analyze lower “start germ numbers,” a certain germ number must first be exceeded for an effect to be measured. The original concentration can be calculated back correspondingly via the time required for reaching this threshold. With very low germ contents, a waiting period of usually from 24 to 48 hours must pass in order to obtain a valid result.

The “ATP method” (ATP=adenosine triphosphate) operates within a similar germ number range and measures the bioluminescence emitted in the course of a biochemical degradation reaction by the ATP of the germs.

All three above-mentioned methods, namely the plate culture method, impedance method and ATP method, can detect only live, vital germs. They are all based on the fact that germs propagate and have a functioning metabolism.

Flow cytometers are technically even more complicated apparatuses—and therefore also markedly more expensive to buy. These instruments pump the germ—containing solution to be analyzed through a very thin capillary. The diameter of said capillary is in parts within the lower micrometer range so that it is possible here to observe individual germs and cells. The dye-labeled germs are excited with high-energy, ideally monochromatic light (e.g., with a laser) at these bottlenecks to produce fluorescence. The intensity of the emitted fluorescent light is usually measured by a photomultiplier (PMT) and subjected to a pulse amplitude analysis (cf. S. Rapposch et al., “Influence of Fluorescence of Bacteria Stained with Acridine Orange on the Enumeration of Microorganisms in Raw Milk,” J. Dairy Sci., 83 (2000), pages 2753 to 2758). With a suitable selection of dyes, it is possible to determine with the aid of said flow cytometers both live/active and dead germs. These flow systems usually cannot be used with samples which have relatively high viscosity and tend to produce foam: first, the capillary is readily blocked and, second, correct detection with the aid of the laser and the PMT is not possible due to the bubbles caused.

In the more recent past, optical fluorescence methods have increasingly replaced the conventional “rapid detection methods” and culturing methods. One method which is the basis of the invention described herein is the Direct Epifluorescent Filter Technique, referred to as DEFT. The Direct Epifluorescent Filter Technique (DEFT) provides a direct method which also allows quantitative “live/dead” germ detection in less than one hour. This unspecific, optical fluorescence method has been known as a qualitative method in university-based basic research for more than 25 years (see, for example, Pettipher et al., Appl. Environ. Microbiol. 44(4): 809-3, 1982) and has established itself increasingly as a quantitative method of investigation in industrial applications (e.g., breweries, dairies, food industry, etc.) since the beginning of the nineties (Hermida et al., J. AOAC Int. 83(6): 1345-1348, 2000 and Nitzsche et al., Brauwelt, No. 5, 177-178, 2000). Moreover, there exists a European regulation for examining irradiated food using a DEFT screening method (EN 13783: 2001 “Detection of irradiation of food using epifluorescent filter technique/aerobic mesophilic germ count (DEFT/APC)—screening method”).

Alternative fluorescent dyes with selective action are supplied as laboratory kits for vital detections by various manufacturers (e.g., by the companies Molecular Probes and EasyProof Laborbedarf GmbH). Some suppliers (e.g., Chemunex) sell device systems; for example, the systems supplied by Chemunex are based either on the principle of flow cytometry (L. Philippe, SOFW Journal 126, 28-31, 2000), which, for the testing of low germ contents, requires an enrichment phase of 24 hours, or on a microscopic filtration method which, however, does not allow any “live/dead” differentiation (Wallner et al., PDA J. Pharm. Sci. Technol. 53(2): 70-74, 1999).

The basic idea of the DEFT method consists of staining germs with fluorescent dyes, causing a fluorescence in said germs and measuring the emitted fluorescent light. The decisive advantage of the DEFT method over the ATP and impedance methods is the fact that the germ-containing sample is filtered prior to or after staining. This enrichment step can reduce the detection limit described also in the literature to one cfu per ml. In addition, both live and dead germs can be stained with the aid of the DEFT method. The detection of dead germs especially is very important in assessing the hygienic status of production plants (e.g., biofilm formation). Moreover, the analytical procedure, i.e., filtration and staining, can be carried out within approximately 30 minutes. The filterability of the product to be investigated is the basic requirement for the DEFT method.

The fluorescent radiation emitted by the germs can be detected in various ways: the most widespread method is the use of a fluorescence microscope. The membrane filter containing the stained germs is scanned field of view by field of view. This involves recording and adding up the number of stained germs by the microscope user with the naked eye. This procedure requires first a large amount of microscopic and microbiological experience, since it is often very difficult for the non-expert to clearly differentiate between actual germs and inevitably occurring foreign particles of similar size and fluorescence. In addition, this method makes high demands on the ability of the user to concentrate because the germs appear at different depth levels on the membrane filters which are not perfectly planar, and overlapping fields of view must be clearly recognized without double counting.

In the past, the aim of automating this evaluation procedure and thereby virtually eliminating mistakes by the user has been approached in the past by altering the following components of a fluorescence microscope: the manually adjustable specimen holder table was replaced by a controllable x, y, z unit which can position a specimen in an automated manner and with exact definition. Instead of the eyepiece which serves the microscope user to view the object, various camera systems are employed. The latter, on the one hand, make visual inspection on a display possible and, on the other hand, there are digitized data available which can subsequently be analyzed, for example with the aid of image recognition algorithms. Other changes also arose on the part of the light sources for fluorescence excitation: in addition to white light sources, use is increasingly made of powerful lasers; the latter have the advantage of a very defined excitation light wavelength, as a result of which optical filters can virtually be dispensed with.

EP 0 713 087 A1 describes a simpler method which, although based on the DEFT method, does not need a microscope. EP 0 713 087 A1 describes a design and a method which comprises stained germs located on a solid support material. The latter is line-scanned by a laser, the emitted fluorescence being detected in the case of at least one wavelength. In comparison with classical microscopy, this method makes possible a markedly quicker analysis which, in addition, is fully automated. On the basis of the described evaluation algorithm, this method, while not being an imaging method in the actual sense, is capable of counting particles, determining their size and, by using the spectral characteristics of the individual particles, detecting absolute germ numbers of less than 100.

A publication by Applied Spectral Imaging, Inc., describes a method for analyzing FISH samples (FISH=Fluorescence in situ hybridization), which can be described as a kind of “location-resolved spectroscopy” (cf. D. G. Soenksen et al. “Multicolor FISH using a novel spectral bio-imaging system,” Proceedings of SPIE (International Society for Optical Engineering), 2678 (1996), pages 303 to 309). A dye-labeled sample is screened with the aid of a fluorescence microscope, and the image is analyzed pixel-by-pixel by a combination of Fourier-transform-(FT) spectroscopy with a CCD camera. Thus, each pixel of the digital image produces a complete spectrum from the ultraviolet range to the near infrared range. Contrary to methods which operate with different filters (see, for example, also I. Ravkin et al. “Automated microscopy system for detection and genetic characterization of fetal nucleated red blood cells on slides,” Proceedings of SPIE (International Society for Optical Engineering), 3260 (1998), pages 180 to 191), a much larger number of wavelengths is thus available for differentiating between germs and autofluorescence/foreign particles. In addition, FT spectroscopy enables the samples to be observed rapidly. However, the huge amount of data and the complex analysis thereof are disadvantageous.

The method of studying cells, described in I. Ravkin et al., loc. cit., has only been automated for acquiring the images; the image material is subsequently evaluated by the user. A filter wheel is used for better distinction of the fluorescent light emitted by the particles. This filter wheel may be configured depending on the fluorescent dyes chosen for staining. Moreover, this reference discusses a possible auto-focusing method.

J. Pernthaler et al. “Automated Enumeration of Groups of Marine Picoplankton after Fluorescence in situ Hybridisation,” Appl. Environ. Microbiol. 69 (2003), pages 2631 to 2637 is also concerned with the aspect of auto-focusing which is important for a successful automation in the analysis of germ-containing samples on solid supports such as membrane filters. The authors propose a “double auto-focusing strategy” for finding the focal point. In a first step, the rough focal point is first/adjusted using “normal” incident light between two filter pieces independent of one another. Auto-focusing of various fields of view of the same filter takes place solely under fluorescence conditions.

WO 2002/064818 A1 relates to an extremely simple procedure for detecting microorganisms: germs are analyzed in two different structural embodiments. First, a sample admixed with a vital/dead dye mixture is applied in drops to a transparent glass support, and fluorescence is recorded by a CCD camera after a 220 magnification. In the second embodiment, the sample is, similarly to the flow cytometry, pumped through a capillary, irradiated with light, and the fluorescence signal generated is recorded using a photodiode or a photomultiplier. In spite of the not particularly differentiated reflection on the problems of automated analyses, mentioned in the above-cited references, the authors in this application propose also LEDs (light-emitting diodes) as a replacement for an Xe lamp for irradiating the sample. Compared with “conventional” light sources, LEDs have the advantage of high stability in the emitted light output. Furthermore, the still existing disadvantages regarding the intensity of LEDs are compensated for by new developments almost every year.

Especially FISH samples often require the use of many different fluorescent markers. If different DNA parts are to be differentiated unambiguously, usage of a plurality of dyes is essential. Since individual filter wheels or filter cubes are unsuitable in these cases, two or more filter cubes are configured in such a way that at least one filter is identical in both. The mathematical method discussed now in WO 98/17992 A2, taking into account two identical filters in two different cubes, enables the data sets obtained to be aligned, especially with respect to the intensity of the emitted fluorescent light. This ensures comparability of images using different filter cubes.

A. Pernthaler et al. “Comparison of Fluorescently Labeled Oligonucleotide and Polynucleotide Probes for the Detection of Pelagic Marine Bacteria and Archaea,” Appl. Environ. Microbiol., 68 (2992), pages 661 to 667, indicate an important phenomenon that plays a part in the correct determination of the germ number, namely bleaching of the dye. This reference describes a study of plankton samples admixed with various fluorescent markers and counted, which study demonstrates that supposed low contents can be explained by more rapid bleaching of a dye in comparison with other fluorescent probes.

U.S. Pat. No. 6,122,396 A combines some of the above-mentioned device and software solutions into one design. The combination of fluorescence microscope, LED illumination and video camera is coupled with a specific image analysis algorithm. A reference (“training”) data set has been deposited in this algorithm, on the basis of which set microorganisms and foreign particles can unambiguously be distinguished. Parameters characterizing this reference data set are morphology and “brilliance” of fluorescence radiation within a particular wavelength range.

When studying living cells by using dyes in the past, the FLIM method (fluorescence lifetime imaging microscopy) has been the center of interest. Similar experiments may also be carried out using FISH samples rather than whole cells. H. J. Tanke et al. “Use of Platinum Coproporphyrin and Delayed Luminescence Imaging to Extend the Number of Targets FISH Karyotyping,” Cytometry 33 (1998), pages 453 to 459 describe a method which involves inferring ultimately the presence of particular DNA fragments from measuring the fluorescence lifetimes—i.e., actually measuring a luminescence—of different dyes. Depending on the hybridization, intensity and lifetime values characteristic for DNA fragments are obtained in this way.

The “Membrane filter Microcolony Fluorescence” method (MMCF method, see, for example, J. Baumgart, Mikrobiologische Untersuchung von Lebensmitteln [Microbiological testing of food], Behr's Verlag 1993, 3rd edition, pages 98 ff.) provides for the preparation of the sample or of the germs present in the sample on a membrane filter. Disadvantageously here is the fact that time-consuming primary enrichment of the germs has to be carried out first, the membrane filter for subsequent epifluorescence microscopy must be pretreated (wetting with special media, dimensioning and drying), and the germs must be counted by counting the fluorescently labeled colonies in an epifluorescence microscope or under a UV lamp.

Although some of the above-described technologies produce a result within a few hours, they are usually very complex with regard to the detection devices required and the necessary knowledge of the user. For this reason, these methods have not been established sufficiently in routine usage up to now. Furthermore, the individual methods have restrictions with regard to specificity and sensitivity. In addition, the germ detection limit of some of the abovedescribed methods is too high so that preceding culturing can often not be dispensed with.

The conventional culturing methods and “rapid detection methods” with corresponding enrichment steps moreover have fundamental disadvantages due to the mode of operation: the selection of nutrient media plays an important part in deciding which microorganisms can be propagated. Selective nutrient media are advantageous here. But even they can propagate only those microorganisms which are physically capable thereof. However, according to most recent knowledge, only 5% of microorganisms can be cultured. Therefore, the conventional methods often produce inaccurate, negative results, although the sample actually contains germs. In addition, the meaningfulness of the analysis is limited by the time available. After the enrichment period has ended, all germs must have propagated to such an extent that they have become visible. Reduced growth due to unfavorable sampling or growing conditions may therefore produce wrongly negative results. Therefore, carrying out the conventional culturing methods for detecting bacteria and fungi often require several days but, as a result, the microbiological results often occur too late to still intervene in the production process in a regulatory manner. Detection methods by means of culturing the germs can be used for detecting only vital germs capable of propagation. However, contaminants have frequently been destroyed by existing product preservation. However, the “dead” germs in the product cannot be detected in this manner and, as a result, possible hygienic problems during production or bottling are not noticed or are noticed only if faults occur, i.e., after preservation failure.

Detecting very low germ contents, in particular below 100 cfu per ml, requires either a waiting period in the range of several hours (impedance or bioluminescence methods) or days (plate culture method) or microscopic methods which are tedious, expensive and tiring (especially for the human eye) and which require instructed skilled personnel.

Automated detection procedures for very small numbers of germs have partly been implemented previously, but they have disadvantages. The analysis of a relatively large amount of sample (several milliliters) in flow cytometry requires a long waiting period, since the capillaries through which the germs flow have corresponding dimensions. Moreover, the use of these capillaries in media with higher viscosity results in blocking, and products containing surfactants make the analysis more difficult due to the formation of foam or bubbles.

The main problem of automated detection of germs on solid supports (e.g., membrane filters) consists first of focusing of the plane of the specimen and second of unambiguous distinction between “actual” germs and inevitably occurring interfering particles with intrinsic fluorescence.

DE 102 59 302 A1, by Applicants themselves, and WO 2004/055203 A1, from the same family of patents, describe a method for quantitatively and/or qualitatively identifying germs in a sample, which comprises a step (a) of sample preparation, with at least some of the germs present in said sample being labeled by means of at least one fluorescent marker, and a step (b) of quantitative and/or qualitative detection and/or evaluation, wherein said detection and/or evaluation carried out in step (b) is by way of fluorescence reflection photometry. Since the fluorescently labeled germs are irradiated only for a relatively short period of time because the measurement data are recorded relatively quickly by fluorescence reflection photometry, a “bleaching effect” of the fluorescent marker and thus a distortion of the measurement result are essentially avoided, although said method does not always enable extremely low germ concentrations to be determined with sufficient reliability, since it is not always possible to discriminate or distinguish foreign or interfering signals sufficiently from the measured signals generated by the fluorescently labeled germs.

### BRIEF SUMMARY OF THE INVENTION

It is therefore the object of the present invention to provide a method of the kind mentioned at the outset, which is suitable for quantitatively or qualitatively identifying germs and which, in particular, avoids at least partially the disadvantages illustrated above, and a corresponding device for carrying out such a method.

According to a first aspect, the present invention therefore relates to a method for quantitatively and/or qualitatively identifying germs in a sample by means of fluorescent labeling and subsequent detection and/or evaluation, said detection and/or evaluation being carried out by way of recording and/or measuring fluorescent emission, wherein the fluorescently labeled germs are subjected to an excitation radiation of a defined wavelength or a defined wavelength range continuously for a defined period of time, and the time course of the fluorescence emission radiation generated due to said excitation in order to be able to discriminate between, first measured signals caused by the fluorescently labeled germs and, second, possible interfering signals.

More specifically, the present invention—according to a first aspect of the present invention—relates to a method for quantitatively and/or qualitatively identifying germs in a sample, comprising a step (a) of sample preparation and a step (b) of detection and/or evaluation, with step (a) comprising labeling at least some of the germs present in the sample by means of at least one fluorescent marker and step (b) comprising a quantitative and/or qualitative detection and/or evaluation, said detection and/or evaluation carried out in step (b) being by way of recording and/or measuring fluorescence emission, where step (b) comprises subjecting the sample containing the fluorescently labeled germs, prepared in step (a), to excitation radiation of a defined wavelength or a defined wavelength range for a defined period of time and recording the time course of the fluorescence emission radiation generated due to said excitation, so that a discrimination is made possible first, between measured signals caused by the fluorescently labeled germs, and second, possible interfering signals, thereby identifying the fluorescently labeled germs in the sample in a quantitative and/or qualitative manner.

### BRIEF DESCRIPTION OF THE SEVERAL VIEWS OF THE DRAWING(S)

Not Applicable

### DETAILED DESCRIPTION OF THE INVENTION

A substantial concept of the present invention can, therefore, be considered that of recording the time course of fluorescence emission radiation with continuous illumination or irradiation by the excitation light source and making possible in this way discrimination first, between measured signals caused by the fluorescently labeled germs, and second, possible interfering signals. This is because, as Applicants have surprisingly found, the measured signals caused by the fluorescently labeled germs have a time course of fluorescence emission, which is different from that of the interfering signals so as to enable in this way the recorded or measured fluorescence emissions to be assigned, i.e., make possible a discrimination first, between measured signals, and second, possible interfering signals. As Applicants have surprisingly found, fluorescently labeled germs usually bleach faster than fluorescent foreign particles and, as a result, this effect can be utilized for assigning the fluorescence emission signals. In this way it is possible to efficiently differentiate first, between the measured signals caused by the fluorescently labeled germs, and second, possible interfering signals, even if the number of germs in the sample is extremely low and consequently the number of the measured signals caused by the fluorescently labeled germs is substantially lower than the number of interfering signals (e.g., by a factor of 1,000 or more). In this way, the method of the invention makes possible relatively simple detection or evaluation with little complexity because this essentially should not require any further processing of the fluorescently labeled germs. Specifically, a time-consuming and complicated step of (pre-)concentration of germs is dispensed with, i.e., detection or evaluation according to the invention provides directly the “authentic” number of germs or number of germs present in the sample.

The term “time course of fluorescence emission radiation,” as used within the scope of the present invention, denotes in particular the time course of the intensity of the fluorescence emission detected or observed. This is not a luminescence phenomenon, since there fluorescence intensity is monitored only after the excitation light source has been switched off.

The term “interfering signals” denotes any detected or measured fluorescence emission signals which are not caused by the fluorescently labeled germs. Such interfering signals may be generated in the sample, for example, by foreign substances having intrinsic fluorescence, but also by unspecific binding of the fluorescent marker to foreign particles or by contaminations due to free fluorescent markers, for example those which have not been washed out.

The method of the invention enables in principle germs in a sample to be identified both quantitatively and qualitatively, i.e., both the type and species of germs per se and their number and concentration to be determined.

Specifically, the method of the invention is carried out in such a way that the time course of the fluorescence emission radiation generated due to said excitation is recorded so as to record the kinetics of the degradation or bleaching of the fluorescent marker or the fluorescently labeled germs (i.e., fluorescent markers bound to the germs when detecting dead germs or fluorescent markers converted due to the metabolism of live germs). As illustrated above, the method of the invention utilizes the surprising finding or fact that the fluorescently labeled germs have a degradation or bleaching behavior of the fluorescent marker, which is different from that of foreign particles, and in particular usually bleach more rapidly than foreign particles. By way of recording the kinetics of the degradation and/or the bleaching of the fluorescent marker, the fluorescence emission signals detected or recorded can—by way of balancing using the interfering signals—be assigned first, to the germs to be determined, and second, to the interfering signals, thereby making possible a specific discrimination or differentiation which enables the fluorescently labeled germs to be determined quantitatively and/or qualitatively in a reliable manner—even at extremely low concentrations.

The kinetics of the degradation and/or bleaching of the fluorescence emission signals or of the fluorescent marker and of the germs labeled therewith are usually recorded by way of recording the time course of the intensities of said fluorescence emission signals.

The number of germs present in the sample can then be determined on the basis of the data determined by fluorescence emission, where appropriate by means of suitable calibration.

The phrase “labeling of at least some of the germs present in the sample” means in particular the following: depending on whether only special germs or types of germs present in the sample or all germs present in the sample are to be determined, only some special germs present in the sample are specifically labeled in the former case (usually with germ-specific fluorescent markers), while in the latter case all of the germs present in the sample are labeled (usually with germ-unspecific fluorescent markers or with a mixture of various germ-specific fluorescent markers).

According to the method of the invention, the number of germs present in the sample can then be determined on the basis of the measured values determined, where appropriate by means of suitable calibration (that is, in the case of fluorescent labeling of all germs present in the sample, the total number of all germs present in the sample and, in the case of fluorescent labeling of only special germs, the total number of the latter). In addition, as will be explained below, the use of germ-specific fluorescent markers also makes possible a qualitative statement regarding the presence of special germs in the sample.

The reproducibility or reliability of the method of the invention can be increased still further by detecting the fluorescence radiation from the processed sample containing the fluorescently labeled germs at wavelengths or wavelength ranges, which differ from one another in each case but are defined in each case in at least two successive time intervals.

The performance of the method of the invention can be increased further by additionally discriminating first, between measured signals caused by the fluorescently labeled germs, and second, by possible interfering signals, by way of analyzing the fluorescence characteristics. This involves in particular recording the ratio of the fluorescence intensities at different but defined wavelengths or wavelength ranges, thereby making possible a reliable discrimination first, between measured signals caused by the germs to be determined, and second, possible interfering signals.

The wavelength or wavelength range of the applied radiation should be matched to the fluorescence characteristics of the fluorescent marker or the fluorescently labeled germs, in particular to their absorption peaks with respect to fluorescence emission.

The performance of the method of the invention can also be increased still further by discriminating—in addition to discriminating by way of recording the time course of the fluorescence emission radiation—by way of the size and/or shape of the emitting particles, and this may be carried out, for example, within the framework of automated image recording processes. Recording the size and/or shape of the emitting particles provides—together with assessing the time course—another criterion for a reliable assignment first, to interfering or foreign particles, and second, to the germs to be determined. Since the germs to be detected are usually smaller than 6 μm, any larger signals can be ignored. The shape must also be seen in this context, since, with the use according to the invention of a 10 times or 20 times magnification microscope lens, the ratio of the major axes in germs is usually approximately 1:1, i.e., the germs appear as round structures—with the exception of the hyphal form of a fungus. In contrast, particles which have a different major axis ratio are thus disregarded.

According to a particularly preferred embodiment of the method of the invention, discrimination first, between measured signals caused by the fluorescently labeled germs, and second, possible interfering signals, is carried out in three stages, i.e., with the application of all three above-mentioned discrimination methods, namely by way of recording the time course of the fluorescence emission radiation and by way of the size and/or shape of the emitting particles and also, finally, by way of analyzing the fluorescence characteristics.

The method of the invention or detection and/or evaluation are carried out by applying or fixing the germs to be determined usually to a support. In this way, at least for the duration of the measurement, the germs to be determined are present in “immobilized form,” insofar as they are in a fixed location on the support and cannot change their position, making a reliable signal assignment possible.

The samples are usually processed as described in the printed publications DE 102 59 302 A1 and WO 2004/055203 A1, by Applicants themselves, which are hereby incorporated in their entirety by reference.

Sample processing carried out in step (a) usually involves applying the fluorescently labeled germs to a preferably porous support (e.g., a membrane filter such as, for example, a polycarbonate membrane filter, or a silicon microsieve), with said support preferably being porous. The support, in particular membrane filter or silicon microsieve, should usually be designed so as to retain the germs or to be impermeable with respect to said germs. For this purpose, the size of the pores of the support should be chosen in such a way that the pore size is smaller than the size of the germs present in the sample. Examples of porous support materials which may be used according to the invention are membrane filters, for example membrane filters based on polycarbonate, PTFE, polyesters, cellulose and cellulose derivatives such as cellulose acetate, regenerated cellulose, nitrocellulose or cellulose mixed esters. Membrane filters suitable according to the invention are sold, for example, by Macherey-Nagel (e.g., the “PORAFIL®” series). Another porous support which may be used is a silicon microsieve which has a particularly smooth and planar surface, as a result of which germs located thereon can be detected even better; furthermore, such sieves are relatively easy to clean and can be used more than once, and, in addition, the silicon microsieves possess good biocompatibility and a rigid structure which gives considerable advantages in their handling.

The use of a porous support, in particular membrane filter or silicon microsieve, advantageously offers the possibility of carrying out detection or evaluation directly on said porous support, in particular without any further sample treatment, sample processing, sample transfer or the like (i.e., in particular without preconcentration).

Advantageously, the fluorescent marker employed in the method of the invention is selected so as to be able to pass through a membrane with regard to the support, in particular membrane filter or silicon microsieve, used in the sample processing carried out in step (a). The advantage here is the fact that no interfering signals caused by excess fluorescent markers and no background noise occur during detection or evaluation, and consequently a favorable signal-to-background ratio or signal-to-noise ratio is achieved.

The germs present in the sample are fluorescently labeled in a manner known per se in step (a) of the method of the invention. This procedure is quite familiar to the skilled worker. For this purpose, for example, the germs to be labeled can be contacted with a solution or dispersion of the fluorescent markers which are in excess with respect to the germs present, with the contacting period having to be adequate in order to ensure complete fluorescent labeling of all germs which ought to be labeled in this step (depending on the selection of the fluorescent marker, for example all germs present in the sample or all germs of only one or more types of germs). After the actual fluorescent labeling, the excess fluorescent markers may then be removed from the fluorescently labeled germs. If a porous support is used, in particular membrane filter or silicon microsieve, whose membrane can be passed with regard to the fluorescent markers but is impermeable with regard to the germs, said removal may be carried out, for example, by discharging (e.g., by applying overpressure or underpressure) the solution or dispersion of the excess fluorescent markers via the porous support and, where appropriate, then rinsing the whole system with water, buffer solutions or other liquids, so that finally only the fluorescently labeled germs (where appropriate together with the germs which have specifically remained unlabeled) still remain on said support. This then enables detection or evaluation to follow immediately in step (b) of the method of the invention.

Where appropriate, step (a) of the sample processing may also comprise inactivating and/or removing germ-inhibiting and/or germicidal substances or components (e.g., preservatives, surfactants etc.) which may be present in the sample. This prevents the later measurement results from being distorted by some of the germs being destroyed or inactivated by said germ-inhibiting or germicidal substances or components during the sample processing or measurement, and therefore an inaccurate, too low a number of germs, from being determined. In this way the number of germs can also be determined in samples containing germ-inhibiting and/or germicidal substances or components (e.g., preservatives, surfactants etc.), enabling the method of the invention to be applied also, for example, to surfactant and dispersion products.

The step of inactivating or removing germ-inhibiting or germicidal substances or components which may be present in the sample is carried out only where appropriate, depending on the type of sample, and is performed advantageously prior to fluorescent labeling, preferably immediately after sampling or directly at the start of the sample processing step (a) of the method of the invention; this guarantees that the germ-inhibiting or germicidal substances, components, ingredients and the like will essentially still have been unable to alter the number of germs present in the original sample. It is equally, albeit with less preference, possible to inactivate or remove germ-inhibiting or germicidal substances or components which may be present in the sample after the fluorescent labeling. It is likewise possible to carry out fluorescent labeling and inactivating or removing the germ-inhibiting or germicidal substances or components at the same time.

The step of inactivating or removing germ-inhibiting or germicidal substances or components which may be present in the sample is carried out only where appropriate, depending on the type of sample, and in a manner known per se. Reference may be made, for example, to the contribution by Stumpe et al. “Chemolumineszenz-basierte Direktnachweise von Mikroorganismen—Ein Erfahrungsbericht aus der Lebensmittel-und Kosmetikindustrie [Chemoluminescence-based methods of directly detecting microorganisms—a report from the food and cosmetics industries]“on pages 317 to 323 of the meeting volume “HY-PRO 2001, Hygienische Produktionstechnologie/Hygienic Production Technology,” 2. Internationaler FachkongreB und Ausstellung, Wiesbaden, May 15-17, 2001, and the literature cited in this contribution; the entire contents of said contribution, including the contents of the literature mentioned therein, are hereby incorporated by reference. This may usually be carried out by contacting the sample to be analyzed with a suitable inactivating and/or conditioning solution. Such inactivating or conditioning solutions are known per se to the skilled worker (e.g., aqueous TLH conditioning solution TLH=Tween-Lecithin-Histidine). Aqueous inactivating or conditioning solutions which are suitable according to the invention may contain, for example, in addition to TLH (e.g., Polysorbate 80=Tween 80, Soya lecithin and L-Histidine) and also buffer substances (e.g., phosphate buffers such as hydrogen phosphate and/or dihydrogen phosphate), other salts (e.g., sodium chloride and/or sodium thiosulfate) and tryptone (peptone from casein).

An inactivating or conditioning solution which is particularly suitable according to the invention has the following composition:

| | | |
|---|---|---|
| Tryptone | 1.0 | g |
| Sodium chloride | 8.5 | g |
| Sodium thiosulfate pentahydrate | 5.0 | g |
| 0.05 M phosphate buffer solution | 10 | ml |
| TLH water | ad 1,000 | ml. |

The TLH water which may be used according to the invention has in particular the following composition:

| | | |
|---|---|---|
| Polysorbate 80 (Tween 80) | 30.0 | g |
| Soya lecithin | 3.0 | g |
| L-Histidine | 1.0 | g |
| Demineralized water | ad 1,000 | ml. |

The phosphate buffer solution which may be used according to the invention has in particular the following composition:

| | | |
|---|---|---|
| Potassium dihydrogen phosphate | 6.8045 | g |
| Dipotassium hydrogen phosphate | 8.709 | g |
| Demineralized water | ad 1,000 | ml. |

The selection of fluorescent marker(s) is not critical. Depending on the application and type of germs, the fluorescent markers known per se from the prior art may be used here, as long as they are suitable for usage within the scope of the method of the invention.

The term “fluorescent marker” has a very broad meaning for the purposes of the present invention and means, in particular, any fluorescent marker which is designed so as to interact with the germs, for example bind to the germs, in particular to their cell wall (envelope) and/or nucleic acid, and/or be absorbed, in particular metabolized and/or enzymatically converted, by said germs.

The fluorescent marker employed according to the invention may be, for example, a germ-unspecific fluorescent marker or a mixture of germ-unspecific fluorescent markers. This enables all germs present in the sample to be fluorescently labeled in a relatively cost-effective manner and thus the total number of germs in the sample to be determined relatively quickly.

A germ-specific fluorescent marker or a mixture of fluorescent markers with different germ specificity may be employed in particular, if selectively only special germs are to be recorded qualitatively and quantitatively.

Equally it is possible to employ a mixture of germ-unspecific and germ-specific fluorescent markers as fluorescent markers.

For example, it is also possible to employ a fluorescent marker which interacts with live germs as fluorescent marker. Equally it is also possible to employ a fluorescent marker which interacts with “dead” germs as fluorescent marker.

It is likewise possible to employ a mixture of fluorescent markers interacting with live germs and fluorescent markers interacting with “dead” germs as fluorescent marker. In this way it is possible to achieve a live/dead differentiation of the germs present in the sample. Such mixtures are known per se from the prior art (see, for example, Stumpe et al., loc. cit., and the system from EasyProof Laborbedarf GmbH, Voerde, Germany, mentioned therein).

The above-mentioned marker system from EasyProof Laborbedarf GmbH was originally introduced for the brewing industry (Eggers et al., Brauindustrie 6, 34-35, 2001). It involves a nonfluorescent precursor of a fluorescent marker being absorbed into an intact microbial cell and converted due to enzymatic activity (esterase) inside the cell (in the cytoplasm) to a fluorescent compound (green coloring=live detection); for this process to be able to occur, an intact cell membrane with membrane potential must be present. “Dead” cells are detected by way of intercalating a specific fluorescent dye into the DNA of the cell. This incorporation in turn can take place only in cells which have a defective cell membrane (red coloring=dead detection). Since both reactions are based on different principles, the results are independent of one another. This labeling technique does not harm the cells, thus enabling the current microbiological status of the sample to be determined in the evaluation.

It is also possible to use, in the method of the invention, for example, the fluorescent markers usually used for labeling germs in epifluorescence microscopy or in DEFT (Direct Epifluorescent Filter Technique) or in MMCF (Membrane filter Microcolony Fluorescence method) as fluorescent markers.

For example, it is possible to employ as fluorescent marker a fluorescent dye or a precursor of such a fluorescent dye from which said fluorescent dye is generated due to interaction with the germs, in particular due to metabolizing and/or enzymatic conversion.

Examples of such precursors of fluorescent dyes are described, for example, in WO 86/05206 A1 and in EP 0 443 700 A2, whose entire particular disclosure content is hereby incorporated by reference (e.g., nonfluorescent diacetylfluorescein which can be converted enzymatically to give fluorescein).

Examples of fluorescent dyes which may be used as fluorescent markers according to the invention are, without limitation, for example 3,6-bis[dimethylamino]acridine(acridine orange), 4′,6-diamido-2-phenylindol (DAPI), 3,8-diamino-5-ethyl-6-phenylphenanthridinium bromide(ethidium bromide), 3,8-diamino-5-[3-(diethylmethylammonio)propyl]-6-phenylphen-anthridinium diiodide(propidium iodide), rhodamines such as rhodamine B and sulforhodamine B and fluorescein thiocyanate. For further examples, reference may also be made to EP 0 940 472 A1 or to Molecular Probes' Handbook of Fluorescent Probes and Research Chemicals, 5th edition, Molecular Probes Inc., Eugene, Oreg. (P. R. Haugland, editor, 1992), whose respective entire disclosure content is hereby incorporated by reference. Furthermore, reference may also be made to the relevant chemicals catalogs (e.g., catalog Biochemicals and Reagents for Life Science Research from Sigma Aldrich, “Fluorescent Labeling Reagents,” edition 2002/2003).

It is also possible to use in the method of the invention, for example, nucleic acid probes (e.g., germ-specific nucleic acid probes) which themselves are fluorescently labeled, in particular with a fluorescent group or a fluorescent molecule. Said fluorescent group or said fluorescent molecule may be bound, for example, covalently or otherwise to the nucleic acid probe. The nucleic acid probe used according to the invention as fluorescent marker may be, for example, a fluorescently labeled oligo- or polynucleotide or a fluorescently labeled DNA probe or RNA probe. Generally, preference is given according to the invention to DNA probes for stability reasons.

Examples of nucleic acid probes which may be used according to the invention as fluorescent markers are, for example, the probes mentioned in WO 01/85340 A2, WO 01/07649 A2 and WO 97/14816 A1 whose particular entire disclosure content is hereby incorporated by reference.

Thus it is possible, for example, to employ as nucleic acid probes the nucleic acid probes usually used for labeling (DNA labeling or RNA labeling) in Fluorescence in situ Hybridization (FISH). For further details in this regard, reference may be made to RÖMPP Lexikon Biotechnogie und Gentechnik, 2nd edition, Georg Thieme Verlag Stuttgart, Germany, pages 285/286, keyword: “FISH,” and the literature cited therein, and also to WO 01/07649 A2, whose particular entire disclosure content is hereby incorporated by reference.

Equally it is also possible to use as fluorescent markers a particularly germ-specific antibody which itself is fluorescently labeled, in particular with a fluorescent group or a fluorescent molecule, wherein said fluorescent group or said fluorescent molecule may be bound covalently or otherwise to said antibody.

The skilled worker will adjust the amount or concentration of fluorescent markers used to the particular circumstances of the individual case. This will be readily familiar to him. For example, in order to stain the germs present in the sample well for a live/dead differentiation, with weak “background staining” at the same time, a suitable “live dy”/“dead dye” mixing ratio should be chosen; selecting said ratio in the individual case is within the ability of the skilled worker.

The detection limit in the method of the invention with regard to the germs to be determined is usually ≦100 Colony-forming units (cfu) per milliliter of sample volume, preferably ≦10 Colony-forming units (cfu) per milliliter of sample volume. The method of the invention therefore does not need any preconcentration step. The low detection limit is of crucial importance, for example, in order to meet particular guidelines or regulations. Thus, for example, according to the CFTA guideline for cosmetic raw materials, a time-consuming and expensive test for the absence of particular problem germs, i.e., pathogenic germs, must be carried out if the germ number limit is markedly higher (e.g., 10² to 10³ cfu/ml).

Generally, the method of the invention can determine germ numbers in the range from about 10 cfu per milliliter of sample volume or even less to about 10⁸ cfu per milliliter of sample volume. For the purposes of quantitative evaluations, the sample should, above a particular number of germs (usually above approximately 10² cfu per milliliter of sample volume), be diluted accordingly, i.e., in a suitable manner, beforehand.

The method of the invention is suitable in principle for determining any germs, in particular pathogenic germs of any kind (e.g., microorganisms of any kind, in particular unicellular microorganisms such as bacteria and fungi, e.g., yeasts or molds).

The method of the invention is suitable in principle for the quantitatively and/or qualitatively identifying germs in any products (i.e., media, matrices, solutions etc.), preferably filterable, in particular liquid and/or free-flowing, products. Solid products or products which are not filterable as such must be transformed during sample processing into a form accessible to the method of the invention; this may be carried out using methods known per se, for example by transfer into a solution or dispersion, crushing, extraction etc.

For example, the method of the invention is suitable for the quantitatively and/or qualitatively identifying germs in food, surfactant-containing products such as detergents and cleaners, surface-treatment agents, dispersion products, cosmetics, hygienic products and body care products, pharmaceuticals, adhesives, cooling lubricants, paints and (paint) coagulations and also raw materials and starting materials for the aforementioned products.

The method of the invention is therefore suitable for any kinds of possible raw materials, intermediate and final products of different fields, such as, for example, food, proprietary goods, cosmetics, adhesives, cooling lubricants (e.g., oily cooling lubricant emulsions); process fluids from plants etc., with the reservation that the germs to be detected should be able to be removed by a separation method, such as filtration or sedimentation. It is also not important here, whether the products are in a solid or liquid form.

Owing to the fact that the method of the invention can be carried out in a relatively simple manner and to the particular combination of method steps, said method is particularly suitable for automation (e.g., within the framework of production control and/or quality control). Aside from production control and/or quality control (e.g., when bottling liquid surfactant products, dispersions, preserved products etc.), the method of the invention is also suitable, for example, for investigating faults or contaminations, for determining the germ status or for evaluating measures for product redevelopment, but also for optimizing or testing plant cleaning processes (e.g., in plants for preparing preserved products), for example within the framework of CIP processes (Cleaning in Place) and SIP processes (Sterilization in Place).

The method of the invention is usually carried out as follows:

The sample may be processed as described in DE 102 69 302 A1 and WO 2004/055203. This may be carried out as follows, for example:

The sample containing the germs to be determined quantitatively and/or qualitatively is introduced to a suitable sample vessel whose bottom has been provided with a usually porous support, for example a membrane filter or a silicon microsieve, and which should be sealable in a germ-free manner. The outside edge of the porous support rests on the sample vessel in such a way that the outside, concentric edge is not occupied by germs. If a sample having germ-inhibiting or germicidal substances or components (e.g., preservatives or surfactants) is to be tested, said substances or components are first inactivated and/or removed by contacting said sample with a suitable inactivating and/or conditioning solution, for a period of time which is sufficient in order to enable said substances or components to be inactivated and/or removed. The inactivating and/or conditioning solution is removed via the membrane filter by means of over or underpressure. Subsequently, excess or remaining inactivating and/or conditioning solution is removed via the membrane filter, where appropriate, by washing once or several times with water, usually by applying an over or underpressure so that the wash water is also removed in a simple manner. This is followed by labeling at least some of the germs present in the sample by means of at least one fluorescent marker. For this purpose, the germs may be contacted, for example, with a solution or dispersion of the fluorescent marker for a time sufficient for labeling said germs. The excess solution or dispersion of the fluorescent marker is then removed via the porous support by applying again an over or underpressure. Finally, the sample may, where appropriate, be subjected to washing once or several times with water, buffer solutions or other liquids, in order to remove excess fluorescent marker. After the water has been removed via the porous support by applying over or underpressure, the porous support may then finally be removed from the sample vessel, resulting in a porous support, in particular membrane filter or silicon microsieve, occupied by fluorescently labeled germs. Said support may undergo measurement or detection directly, i.e., usually without further processing of the sample or treatment of the sample or of the filter. The measurement comprises irradiating the support occupied by fluorescently labeled germs then with light of suitable wavelength and in the process the support is scanned. The data determined correlates with the number of germs on the membrane filter and in the sample, respectively.

The measurement design on which the present invention is based includes a few “deviations” from a usual fluorescence microscope and, at some points, goes a step further.

The detector may comprise, for example, the following components: a specimen holder table which can be moved in all three spatial directions. The step size of its positioning is, for example, about 2 μm. The table is controlled, for example, with the aid of a computer, with the parameters for scanning a support or membrane filter or silicon microsieve being deposited within a corresponding measurement program. These parameters describe automated focusing, the scanning width in the x and y directions, the depth profile in the z direction and the area of the sample to be tested. The specimen holder table itself may be shaped, for example, in such a way that preferably standard slides measuring 26 mm in width and 76 mm in length (ISO standard 8037/1) can be fixed firmly.

The sample is irradiated, for example, by an LED (light-emitting diode). Using an LED has the advantage that the specimen to be studied is exposed to only little thermal stress and thus preserved for a longer time. The excitation wavelength of the LED depends in particular on the absorption peak of the fluorescent dye used. The LED light is directed via a dichroitic beam splitter to the sample. The fluorescence radiation emitted by the germs may be spectrally filtered on its path through a microscopic lens to a CCD camera. This enables the fluorescence radiation to be detected in a wavelength-dependent manner; basically, a “spectral fingerprint” of the germs is obtained.

Automated focusing in different, particular planes can be carried out with the aid of an auto-focusing function. The image sections obtained in this way are approximately 1.5 mm×1.0 mm in size, for example, with preference being given to utilizing a microscope lens with tenfold magnification. The auto-focusing function is used for defining a particular image plane. Starting from the latter, the specimen to be studied is moved in the z direction (i.e., vertically to the area of the specimen) in defined steps. In this way, images in various depth planes are recorded. Subsequent projection of the image planes finally delivers an overall image with depth of field.

In the overall analysis of a support or membrane filter or silicon microsieve, a plurality of fields of view (e.g., in the preferred size of 1.5 mm×1.0 mm) in various depths using different optical filters thus provide a relatively large amount of data. It is further possible to record a number of image sequences in a particular field of view with an established depth and an optical filter at a relatively long exposure time.

The images generated in this way are assessed for their actual germ content, taking into account various evaluation parameters. First, the size and shape of a detected particle plays an important part. Since the germs to be detected are usually smaller than 6 μm, any larger particles can be ignored. The shape must also be seen in this context; this is because, with the preferred magnification mentioned herein, the ratio of the major axes of the germs is, in a first approximation, 1:1, i.e., the germs appear as round structures (with the exception of the hyphal form of a fungus). Particles which have a different major axis ratio are thus disregarded.

An additional parameter is the analysis of the fluorescence characteristics. Of interest herein is not only the intensity of the radiation at a particular wavelength or wavelength range. Rather, the ratio of the fluorescence intensities at different wavelengths/wavelength ranges is decisive for discriminating between germs and other particles. These wavelength differences are additionally depicted in the above-mentioned depth-of-field image as false colors. The excitation light is removed using a dichroitic beam splitter with a “cuton” at 500 nm. This results in the excitation light being reflected virtually completely at the filter, while the fluorescent light emitted by the stained live germs passes virtually completely above a wavelength of 520 nm. A long pass filter with a “cuton” at 520 nm is arranged in the fluorescent light path in order to attenuate the intensive excitation light of the LED even further. All other filters are integrated into a filter wheel, with one position (“position 1”) of the filter wheel remaining without filter; this position is used for measuring the total fluorescent light emitted by the sample. Bandpass filters which filter out the green fluorescent light with different widths are used at two further positions (“positions 2 and 3”). Another three filters at the remaining positions of the filter wheel (“positions 4 to 6”) are long pass filters with “absorbence cutons” shifted further and further into the red range.

Finally, the fluorescence intensity determined for a particular irradiation time is evaluated. This is not a measurement of the fluorescence lifetime, which involves detecting the duration of the “afterglow” with a single excitation. Rather, the present invention comprises illuminating a field of view for a particular time, taking an image of said field, illuminating again, etc. This bleaching generated using the LED serves in a decisive way to differentiate germs and other particles unimportant for determining the germ number (“interfering particles”). Since germs absorb only a finite amount of fluorescent dye, the latter is bleached due to irradiation of the LED after a particular time; the germs glow only weakly or do not grow any more at all. However, the intensity of particles which have not been stained by the fluorescent dye and which cause a fluorescence due to intrinsic properties (e.g., plastic particles) remains virtually unaffected by the irradiation time.

The system used according to the invention is capable of focusing on an observing field of view in a fully automated manner. In order to produce an image of the germs and other particles present on the membrane filters, which is as accurate as possible, auto-focusing is repeated after each step or in each field of view.

With a relatively long illumination time, there is a fundamental difference in the fluorescence intensity between germs stained with fluorescent dyes and interfering particles, i.e., particles having an intrinsic fluorescence. In the case of the germs, the dye is gradually bleached by the high-energy light. However, in the case of particles which fluoresce due to their intrinsic properties (intrinsic fluorescence), a relatively long illumination does not cause any change in intensity or causes only a very slight change. This criterion is very important if particles of equal size and of similar spectral characteristics are to be distinguished from one another. In this connection, it should be noted that this described bleaching method for differentiation cannot be equated with the fluorescence lifetime studies (cf., e.g., H. J. Tanke et al., loc. cit.).

The combination of the characteristics shape/size and spectral properties and also bleaching behavior of particles—both germs and “interfering particles”—results in an extremely high reliability in differentiation or discrimination. A subsequent microscopic inspection by the user is therefore no longer required. In this way it is possible to carry out automated analyses in the range of <10 KBE/ml in a reproducible and reliable manner.

A multiplicity of advantages are associated with the method of the invention, of which the important ones are listed below, but without limitation:

One advantage of the method of the invention can be seen in the fact that it can be carried out in an automated manner. The complete automation of the entire process enables the method to be carried out in a simpler, quicker and more reproducible way. This yields advantages with regard to costs, personal requirement and sensitivity. The high reproducibility in carrying out the tests is likewise highly advantageous.

Another advantage of the method of the invention consists of the use of standardized or conventional components: the whole system required for carrying out the method of the invention is integrated in such a way that a multiplicity of standardized or conventional components (vessels, media, filters etc.) can be used, thereby reducing the workload for the operator and increasing the reliability of the method.

Another advantage of the method of the invention consists of the simple detection and evaluation: for example, the method of the invention may be carried out in a suitable sample vessel so that the labeled germs are prepared on a suitable support, for example a filter membrane.

Another advantage of the method of the invention is also the speed of carrying out the method of the invention: the method of the invention allows determination of the germ number even after a few minutes, depending on the type of germs and their number. In contrast, conventional culturing methods require up to several days.

A particular advantage is also the high sensitivity of the method of the invention: the method of the invention allows the determination of germs even at high dilution. Accelerated culturing methods are not sufficiently sensitive for a detection limit of 10 cfu per milliliter of sample volume.

Finally, another advantage of the method of the invention must be seen in the user friendliness: the entire process of determining the germ load of a sample, with automation, consists merely of introducing the sample and starting the process. Subsequently, the user obtains the numerical value of the germ load. The effort for sample processing and measurement is minimal. The system can therefore also be integrated in an ideal manner into process systems for quality monitoring, quality assurance and quality documentation.

According to another, second aspect of the present invention, the present invention also relates to a device as described in claim 35 for quantitatively and/or qualitatively identifying germs in a sample by means of fluorescence labeling or by means of a fluorescent marker, wherein the device is designed in such a way that the fluorescence emission time course for discriminating first, between the measured signals caused by the fluorescently labeled germs, and second, possible interfering signals can be recorded.

The present invention relates in particular to a device for quantitatively and/or qualitatively identifying germs in a sample by a method with sample processing and subsequent detection and/or evaluation, wherein, by means of said device, in the course of sample processing at least some of the germs present in the sample can be labeled by means of at least one fluorescent marker and detection and/or evaluation can be carried out, with utilization of said fluorescent marker, by recording and/or measuring fluorescence emission, in particular for carrying out the above-described method of the invention wherein said device has:
a sample receptacle,
a preferably porous support arranged at an outlet of said sample receptacle, in particular at its bottom, for applying and/or fixing the germs, in particular membrane filter or silicon microsieve, wherein said support is designed so as to retain the germs to be detected and/or to be impermeable with respect to said germs to be detected, and
a detection system which is designed for conducting a fluorescence emission measurement and on which the sample receptacle with the support and/or the support removed from the sample receptacle can be positioned for detection and/or evaluation purposes, wherein said detection system has a, preferably computer-controlled, control and/or evaluation unit as part of said detection system and/or for controlling said detection system, and comprises a source of radiation, by means of which the fluorescently labeled germs can be exposed to an excitation radiation of a defined wavelength or a defined wavelength range for a defined period of time, wherein said detection system, in particular said control and/or evaluation unit, has means for recording the time course of the fluorescence emission radiation generated due to said excitation, in order to enable a discrimination first, between the measured signals caused by the fluorescently labeled germs, and second, possible interfering signals to be recorded.

Further advantageous embodiments of the device of the invention are subject matter of the device subclaims (claims 37 to 40). The comments made with respect to the method of the invention apply accordingly to the device of the invention.

According to a further, third aspect of the present application, the present invention furthermore relates to the use according to the invention of the device of the invention, as described above, as it is the subject matter of the use claims (claims 41 to 43).

Further embodiments, modifications and variations and also advantages of the present invention are readily apparent to, and can be implemented by, the skilled worker reading the description, without thereby leaving the scope of the present invention.

1. A method for quantitatively and/or qualitatively identifying germs in a sample comprising a sample preparation step (a) comprising labeling at least a portion of the germs present in the sample with at least one fluorescent marker and a quantitative and/or qualitative detection step (b) comprising subjecting the sample comprising the fluorescently labeled germs prepared in step (a) to an excitation radiation of a defined wavelength or a defined wavelength range for a defined period of time and recording the time course of the fluorescence emission radiation generated due to said excitation, so that a discrimination is made possible first, between the measured signals caused by the fluorescently labeled germs, and second, possible interfering signals, thereby identifying the fluorescently labeled germs in the sample in a quantitative and/or qualitative manner.

2. The method of claim 1 wherein the kinetics of degradation and/or bleaching of the fluorescent marker are recorded.

3. The method of claim 2 wherein the kinetics are recorded by recording the time course of the fluorescence emission signal intensities.

4. The method of claim 1 wherein the number of germs present in the sample is determined based on the measured fluorescence emission values.

5. The method of claim 1 wherein the sample comprising the fluorescently labeled germs is irradiated with different wavelengths or wavelength ranges in at least two successive (time) intervals whereby differing fluorescence radiation at wavelengths or wavelength ranges is produced and detected.

6. The method of claim 1 wherein the wavelength or wavelength range of the excitation radiation is correlated with the absorption peaks of the fluorescence of the fluorescent marker and/or of the fluorescently labeled germs.

7. The method of claim 1 wherein an additional discrimination is carried out by way of size and/or shape of the emitting particles.

8. The method of claim 7 wherein the additional discrimination is carried out by analyzing the ratio of fluorescence intensities at differing defined wavelengths or wavelength ranges.

9. The method of claim 1 wherein the germs are applied and/or fixed to a support.

10. The method of claim 9 wherein the support has a porous structure wherein the size of the pores of the support is smaller than the size of the germs.

11. The method of claim 1 wherein step (a) comprises applying the fluorescently labeled germs to a porous filter selected from the group consisting of a porous membrane filter, a polycarbonate membrane filter, or a silicon microsieve wherein the filter is impermeable to the germs.

12. The method of claim 11 wherein the pore size is smaller than the size of the germs.

13. The method of claim 11 wherein step (b) is carried out on the support without further sample treatment or sample processing or sample transfer.

14. The method of claim 11 wherein the porous filter is permeable to the fluorescent marker.

15. The method of claim 1 wherein step (a) is further comprised of inactivating and/or removing germ-inhibiting and/or germicidal substances by contacting said sample with a suitable inactivating and/or conditioning solution.

16. The method of claim 1 wherein the fluorescent marker interacts with the germs by attachment to the cell wall of the germs.

17. The method of claim 1 wherein the fluorescent marker is a germ-unspecific fluorescent marker or a mixture of germ-unspecific fluorescent markers.

18. The method of claim 1 wherein the fluorescent marker is a germ-specific fluorescent marker or a mixture of germ-specific fluorescent markers.

19. The method of claim 1 wherein the fluorescent marker is a mixture of germ-unspecific fluorescent markers and a mixture of germ-specific fluorescent markers.

20. The method of claim 1 wherein the fluorescent marker interacts with live germs.

21. The method of claim 1 wherein the fluorescent marker interacts with dead germs.

22. The method of claim 1 wherein the fluorescent marker is a mixture of fluorescent markers interacting with live germs and fluorescent markers interacting with dead germs.

23. The method of claim 1 wherein the fluorescent marker used is a fluorescent marker commonly used for labeling germs in epifluorescence microscopy, in DEFT or in MMCF.

24. The method of claim 1 wherein the fluorescent marker is either a fluorescent dye or a fluorescent dye precursor generated by interaction with the germs.

25. The method of claim 1 wherein the fluorescent marker is a fluorescent dye selected from the group consisting of 3,6-bis[dimethylamino]acridine(acridine orange), 4′,6-diamido-2-phenylindol (DAPI), 3,8-diamino-5-ethyl-6-phenylphenanthridinium bromide(ethidium bromide), 3,8-diamino-5-[3-(diethylmethylammonio)propyl]-6-phenylphenanthridinium diiodide(propidium iodide), rhodamine B, sulforhodamine B and fluorescein thiocyanate.

26. The method of claim 1 wherein the fluorescent marker used is a fluorescently labeled germ-specific nucleic acid probe wherein said nucleic acid probe is a fluorescently labeled DNA probe or RNA probe.

27. The method of claim 26 wherein the nucleic acid probe is a FISH probe.

28. The method of claim 1 wherein the fluorescent marker is a fluorescently labeled germ-specific antibody.

29. The method of claim 1 wherein the detection limit with respect to the germs is <100 colony-forming units (cfu) per millimeter of sample volume.

30. The method of claim 1 wherein the germs are bacteria, fungi or a combination thereof.

31. The method of claim 1 wherein the sample is selected from the group consisting of food products, surfactant-containing products, surface-treatment agents, dispersion products, cosmetics, hygiene products, body care products, pharmaceuticals, adhesives, cooling lubricants, paints and paint coagulations.

32. The method of claim 1 wherein the sample is a liquid and/or free-flowing product.

33. The method of claim 1 wherein the method is carried out in an automated manner.

34. The method of claim 1 wherein the method is employed in production control and/or quality control.

35. A device for carrying out the method of claim 1 comprising means for recording the fluorescence emission time course for discriminating first, between the measured signals caused by the fluorescently labeled germs, and second, possible interfering signals.

36. The device of claim 35 comprising a sample receptacle having an inlet opening for receiving material comprised of germs and an outlet opening for delivering material; a porous support within the receptacle positioned proximal to the outlet for retaining germs labeled with a fluorescent marker and passing at least partially germ-free material through the porous support; means for generating excitation radiation of a defined wavelength or a defined wavelength range; means for directing the excitation radiation incident upon the fluorescent marker-labeled germs retained on the porous support; means for detecting and measuring fluorescence emission radiation generated in response to the excitation radiation; and means for recording the time course of the fluorescence emission radiation.

37. The device of claim 36 wherein the porous support is a polycarbonate membrane filter or a silicon microsieve.

38. The device of claim 36 wherein the size of the pores of the support is smaller than the size of the germs in the sample.

39. The device of claim 36 further comprising a thermostating unit for controlling the temperature of the sample receptacle.
